(19) United States
(12) Patent Application Publication
(10) Pub. No.: US 2009/0082677 A1
(43) Pub. Date: Mar. 26, 2009

(54) EXERCISE ASSISTING DEVICES

(75) Inventors: Fu-Ming SHIH, Hsinchu City (TW); Hsin-Yi PENG, Hsinchu City (TW)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: INDUSTRIAL TECHNOLOGY RESEARCH INSTITUTE, Chutung (TW)

(21) Appl. No.: 11/862,065

(22) Filed: Sep. 26, 2007

(51) Int. Cl. A61B 5/01; A61B 5/02; A61B 5/021; G01C 22/00; G01N 33/20

(52) U.S. Cl. 600/485; 235/105; 436/74; 600/300; 600/508; 600/549

(57) ABSTRACT

An exercise assisting device comprising a detector configured to detect at least one physiological data of a user, a control module configured to convert the at least one physiological data into at least one index in accordance with an algorithm, each of the at least one index being representative of an exercise performance level, a memory module including a first section configured to store a number of predetermined data related to a number of exercise performance levels, a second section configured to store the at least one index, and a third section configured to store the algorithm, and a display module configured to display at least one of the number of predetermined data in accordance with the at least one index.

### BACKGROUND OF THE INVENTION

The present invention generally relates to exercise assisting devices and, more particularly, to devices capable of monitoring the physiological condition of an exerciser and providing interaction with the exerciser or among a group of exercisers.

The benefits of regular and proper exercise to improve overall health, appearance and longevity have been well known. Many millions of people around the world have adopted a fitness lifestyle. They may routinely participate in various exercise activities, such as, for example, jogging, weight training and aerobics, and may take exercise outdoors or use indoor exercise equipment at fitness clubs, gyms, offices and at home. However, outdoor exercise schedules may be boring to keep and indoor exercise equipment may be boring to use for their inability to successfully stimulate a user or exerciser to continue exercising. It may be desirable to have devices that are able to stimulate and encourage an exerciser to keep his or her exercise schedule and continue exercising. It may also be desirable to have devices that are able to interact with an exerciser or allow an exerciser to interact with other exercisers.

### BRIEF SUMMARY OF THE INVENTION

Examples of the present invention may provide an exercise assisting device comprising a detector configured to detect at least one physiological data of a user, a control module configured to convert the at least one physiological data into at least one index in accordance with an algorithm, each of the at least one index being representative of an exercise performance level, a memory module including a first section configured to store a number of predetermined data related to a number of exercise performance levels, a second section configured to store the at least one index, and a third section configured to store the algorithm, and a display module configured to display at least one of the number of predetermined data in accordance with the at least one index.

Examples of the present invention may further provide an exercise assisting device comprising a detector configured to detect at least one physiological data of a user, a network communicating device configured to allow the user to play a game over a communications network, the game including a comparison based on one of the at least one physiological data with that of another user, a memory module including a first section configured to store a number of predetermined data related to a number of exercise performance levels, a second section configured to store the at least one physiological data, and a third section configured to store a program of the game, and a display module configured to display at least one of the number of predetermined data in accordance with a result of the game.

Examples of the present invention may still provide an exercise assisting device comprising a detector configured to detect at least one physiological data of a user, a control module configured to convert the at least one physiological data into at least one index in accordance with an algorithm, each of the at least one index being representative of an exercise performance level, a network communicating device configured to allow the user to play a game over a communications network, the game including a comparison based on one of the at least one index with that of another user, a memory module including a first section configured to store a number of predetermined data related to a number of exercise performance levels, a second section configured to store the at least one index and a result of the game, and a third section configured to store a program of the game, and a display module configured to display at least one of the number of predetermined data in accordance with at least one of the at least one index or the result of the game.

Some examples of the present invention may provide a method of operating an exercise assisting device, the exercise assisting device comprising a detector, a control module, a memory module and a display module, the method comprising detecting at least one physiological data of a user, converting the at least one physiological data into at least one index in accordance with an algorithm, each of the at least one index being representative of an exercise performance level, storing a number of predetermined data related to a number of exercise performance levels, storing the at least one index, and displaying at least one of the number of predetermined data in accordance with the at least one index.

In one aspect, converting the at least one physiological data into the at least one index may further include calculating the at least one physiological data to obtain a set of values, and comparing the set of values with a set of reference values.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings examples which are presently preferred. It should be understood, however, that the invention is not limited to the precise arrangements and instrumentalities shown.

In the drawings:

FIG. 1A is a schematic diagram of an exercise assisting device consistent with an example of the present invention;

FIG. 1B is a block diagram of a processor unit of the exercise assisting device illustrated in FIG. 1A;

FIG. 1C is a schematic diagram illustrating a memory module of the processor unit illustrated in FIG. 1B;

FIG. 2A is a flow diagram illustrating a first mode of the exercise assisting device illustrated in FIG. 1A;

FIG. 2B is a flow diagram illustrating a second mode of the exercise assisting device illustrated in FIG. 1A; and

FIG. 2C is a flow diagram illustrating a third mode of the exercise assisting device illustrated in FIG. 1A.

### DETAILED DESCRIPTION OF THE INVENTION

Reference will now be made in detail to the present examples of the invention illustrated in the accompanying drawings. Wherever possible, the same reference numbers will be used throughout the drawings to refer to the same or like portions.

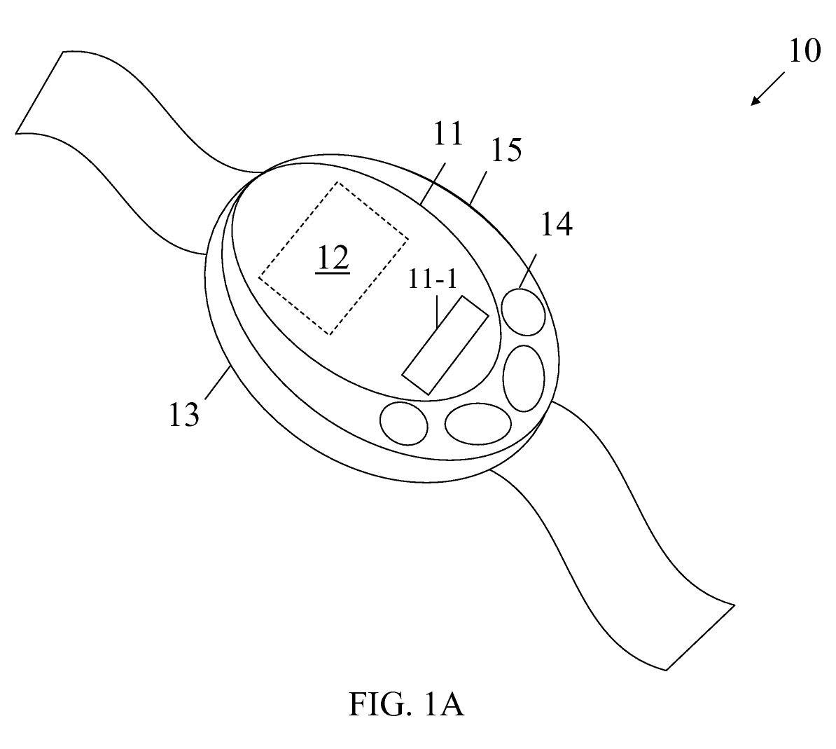

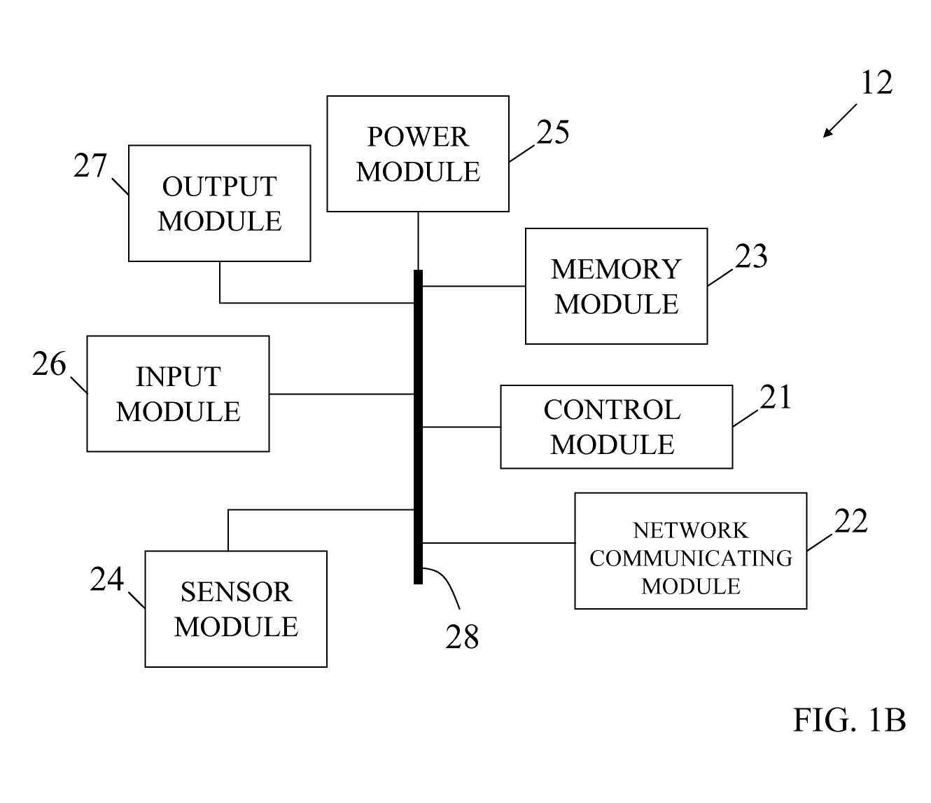

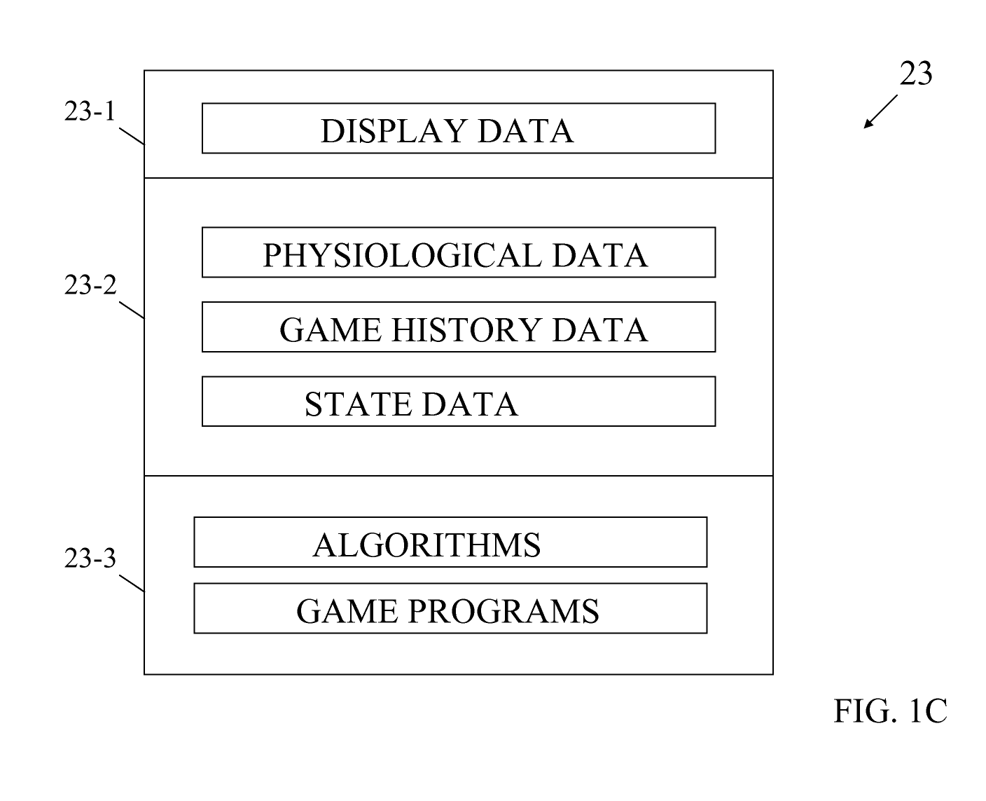

FIG. 1A is a schematic diagram of an exercise assisting device 10 consistent with an example of the present invention. Referring to FIG. 1A, the exercise assisting device 10 may include an output unit 11, a processor unit 12, a detector unit 13 and an input unit 14. The output unit 11 may include a liquid crystal display (LCD) device located on a front side of a housing 15 to facilitate display of visual data such as image, text and optical data. In another example, the output unit 11 may further include a loudspeaker (not shown) to facilitate broadcasting of audio signals such as music or sounds. Furthermore, a section 11-1 may be provided to facilitate display of time information or a physiological condition of a user. The processor unit 12, which may be embedded in the housing 15 under the LCD device, may include circuits or devices required to perform the functions of the exercise assisting device 10. The processor unit 12 will be further discussed below with respect to FIG. 1B. The detector unit 13 may be configured to obtain at least one physiological condition of a user. In one example, the detector unit 13 may include a pulse detector (not shown) capable of detecting the pulse rate of a user. In another example, the detector unit 13 may include a temperature detector (not shown) capable of detecting the body temperature of a user. In still another example, the detector unit 13 may be able to detect the blood pressure or sweat sodium concentration of a user. The input unit 14 may include a number of touch pads or buttons to facilitate a user to select among a number of operation modes, which may include an exercise mode, a game mode and a share mode, and to set or reset a time for an exercise activity. In another example, the input unit 14 may further include a speech recognition device (not shown) capable of converting a speech signal into a sequence of words.

The exercise may include but is not limited to walking, jogging, running, striding, climbing, cycling, dancing, swimming, tennis or badminton play, weight training, aerobics and a sport activity using exercise equipment. In the present example, the exercise assisting device 10 may be implemented in the form a wrist watch. Skilled persons in the art will understand that the exercise assisting device 10 may take other forms for other applications. For example, the detector unit 13 may include a skin-contact type detector capable of measuring at least one of the pulse rate, blood pressure, sweat sodium concentration or body temperature of a user, and may be configured to attach to the wrist of neck of the user. In other examples, the detector unit 13 may include a machine-contact type detector capable of measuring the step counts or walking speed of a user, and may be configured to couple to an exercise machine such as a pedometer.

FIG. 1B is a block diagram of the processor unit 12 of the exercise assisting device 10 illustrated in FIG. 1A. Referring to FIG. 1B, the processor unit 12 may include a control module 21, a network communicating module 22, a memory module 23 and a sensor module 24. The control module 21 may include a microprocessor or a chip, which is configured to provide the functions of, for example, arithmetic and logic operation and program execution. The network communicating module 22 may allow interaction among a group of users based on a networking technique such as, for example, ad hoc or peer-to-peer technique. The ad hoc may refer to a networking connection method for wireless devices, wherein connection may be established between network nodes without the need of a base station. The peer-to-peer technique in one example may include but is not limited to Wireless Fidelity (WiFi), Bluetooth and ZigBee. As an example, WiFi may refer to a brand originally licensed by the WiFi Alliance to describe the underlying technology of wireless local area networks (WLAN) based on the IEEE 802.11 specifications. WiFi may be used for mobile computing devices such as laptops in LANs, and for more services such as the Internet access. Bluetooth may provide a way to connect and exchange information between devices such as mobile phones, laptops, personal computers (PCs), printers, digital cameras, and video game consoles over a secure, globally unlicensed short-range radio frequency. ZigBee may refer to a published specification set of high level communication protocols designed to use small, low power digital radios for wireless personal area networks (“WPANs”). In one example according to the present invention, the network communicating module 22 may include an interface device such as a universal serial bus (USB) to facilitate data communication with a PC or notebook. In another example, the network communicating module 22 may be configured to support the “Global System for Mobile communications” (GSM) or the “third-generation” (3G) services. The network communicating module 22 may thus enable the exercise assisting device 10 to function as a communication node in a communication network.

The memory module 23 may be configured to store data required to perform the functions of the exercise assisting device 10. The memory module 23 may include a flash memory device and will be discussed below with respect to FIG. 1C. The sensor module 24 may monitor the operation of the detector unit 13 and receive physiological information therefrom. The processor unit 12 may further include a power module 25, an input module 26 and an output module 27. The power module 25, which is capable of providing electricity to the exercise assisting device 10, may include but is not limited to an alkaline battery, a lithium battery and a rechargeable battery. The input module 26 and the output module 27 may include circuits or components capable of processing incoming and outgoing signals, respectively. The modules 21 to 27 of the processor unit 12 may communicate over a bus 28.

FIG. 1C is a schematic diagram illustrating the memory module 23 of the processor unit 12 illustrated in FIG. 1B. Referring to FIG. 1C, the memory module 23 may include a first section 23-1, a second section 23-2 and a third section 23-3. The first section 23-1 may include data, such as images and texts, to be displayed through the output module 11. The image data may include but are not limited to graphics and pictures of a virtual character such as a figure or a virtual pet such as a dog, a cat, a monster, a plant or the like. The virtual character or pet may behave or make motions and gestures based on the type of exercise or the physiological condition of a user. The text data may include but are not limited to descriptive and indicative remarks, short slogans and alert words. In one example, the images may include a chicken, which may be “running”, “walking”, “jogging”, “striding”, “mountain climbing”, “cycling”, “dancing”, “listening to music”, “swimming”, “rowing a boat” and “playing badminton”. Furthermore, the texts may include, for example, “Good”, “Sucks”, “Well done”, “Not bad”, “Lazy bone”, “Go for it”, “More exercise”, “You win”, “You lose”, “Keep in good shape”, “High body temperature”, “Too high pulse rate” and “Money can't buy your health”. The display data may be displayed on the output unit 11 under the control of the processor unit 12 based on the physiological condition of the user.

The second section 23-2 of the memory module 23 may include physiological data, game history data and state data. The third section 23-3 of the memory module 23 may include algorithms for determining parameters and indices for the physiological data, and game programs to allow a user to play games with other users. The physiological data may include the physiological condition, such as the pulse rate, sweat sodium concentration and body temperature, of a user detected by the detector unit 13. The game history data in one example may include the result of a game between a user and another user or among a group of users played over the Internet through the network communicating module 22. Furthermore, the game, which may be a mini game, may include a comparison based on scores related to at least one of physiological conditions such as pulse rate, sweat sodium concentration and body temperature. The result of the comparison may be “win”, “lose” or “tie”. The state data may include parameters or indices related to the state of the virtual character or pet. The parameters or indices may be determined by performing the algorithms and game programs in the third section 23-3. Specifically, as an example of pulse rate being a physiological condition, the pulse rate detected while the user is exercising or immediately after an exercise activity may be stored in the second section 23-2 as the “physiological data.” Furthermore, a set of parameters related to the pulse rate may be determined by executing an algorithm in the third section 23-3 and then stored in the second section 23-2 as the “state data.” If the user plays a game with another user by performing a game program in the third section 23-3, a result of the game may be stored in the second section 23-2 as the “game history data.” Subsequently, an image or a text or both related to the current state data or the most updated game history data may be displayed through the output unit 11. Accordingly, the parameters or indices may represent a level of exercise performance performed by the user, and the output unit 11 may provide a signal responsive to the performance level. Moreover, the game program may control the game based on the performance level.

The exercise assisting device 10 may be configured to perform among an exercise mode, a game mode and a share mode. Various outputs or indexes may result from performing these modes. Specifically, outputs X and Rₜ may be related to the exercise mode, while Gₜ and Cₜ may be respectively related to the game mode and the share mode. The outputs X, Rₜ, Gₜ and Cₜ may be related to an exercise performance level based on at least one physiological condition of a user and may be calculated or determined by equations or algorithms described below.

X=f₁( Y̅,P, S̅) (A)

Where Y̅, in the form a vector, may include at least one physiological condition of a user, P may refer to a physical profile such as at least one of the height, weight, sex and age of the user, and S̅, in the form of a vector, may include a set of reference data for intensity or duration of exercise related to at least one physical profile factor, for example, age. X may refer to a value or level decided by the function of Y̅, P and S̅. It is generally believed that an optimal exercise prescription or schedule for competitive training as well as for fitness improvement may be a balance between the frequency, intensity and duration of exercise. To maintain a desirable physical fitness including such as cardiorespiratory fitness and muscular fitness, American College of Sports Medicine (ACSM) recommends 3 to 5 times of exercise a week, 60-90% of maximum heart rate (HR_{MAX}) and 20 to 60 minutes of exercise per time, which may satisfy the requirements for frequency, intensity and duration, respectively. The maximum heart rate may refer to the highest heart rate achieved in an all-out effort. Maximum heart rate may be individual due to heredity, fitness level and age. However, according to ACSM, maximum heart rate may be predicted on the basis of age. For example, maximum heart rate for adults may equal to (220—age). Furthermore, according to the goal of an exercise schedule, however, the target heart rate may be calculated as a percentage of the maximum heart rate. Typically, 50-60% of the maximum heart rate may represent light, 60-70% light to moderate, 70-80% moderate to heavy, 80-90% heavy and 90-100% very heavy intensity. In one example according to the present invention, 50-60% of the maximum heart rate may be related to a goal of an exercise schedule for keeping healthy, 60-70% for weight control, 70-80% for aerobic training, and 80-100% for competitive training.

In addition to maximum heart rate, sodium concentration in sweat may also serve to evaluate intensity of exercise. Sweat sodium concentration of a healthy adult may range from approximately 50 to 65 millimoles per litter (mmol/L). Furthermore, the period of exercise, ranging from approximately 20 to 60 minutes, may serve to evaluate duration of exercise. For a healthy adult of 30 years old having an exercise schedule with a goal to lose or control weight, the S̅ vector may be, for example, (123, 30, 55), which is calculated below.

(1) The maximum heart rate (HR_{MAX}) may be both age dependent and exercise goal dependent. In the present example,

HR_{MAX}(30,60-70%)=(220−30)×(60-70%)=114˜133

By taking the average, HR_{MAX}(30,65%)=123.

(2) The duration of exercise may be identified as thirty (30) minutes, given a healthy adult aged thirty.

(3) The intensity of exercise in term of sweat sodium concentration may be identified as 55 mmol/L, given a healthy adult of thirty practicing exercise for 30 minutes.

The function f₁, in one example according to the present invention, may be defined in an equation below.

$${f}_{1} ( \overline{Y} , P , \overline{S} ) = \frac{{\sum }_{m = 1}^{n} Wm [ 1 - \frac{\vert Ym - Sm \vert}{Sm} ]}{n} ,$$

n and m being natural numbers.

Where Wm is a weight value related to Ym and satisfies

$$\frac{{\sum }_{m = 1}^{n} Wm}{n} = 1 ,$$

and Ym and Sm may refer to an m-th entry of the Y̅ and S̅ vectors, respectively. Given n=3, for the above-mentioned healthy adult, the Y̅ and S̅ vectors may be, for example, (110, 20, 53) and (123, 30, 55), respectively. The value of X may then be calculated below.

$$\begin{matrix}\begin{matrix}X = \frac{[ 1 - \frac{123 - 110}{123} ] + [ 1 - \frac{30 - 20}{30} ] + [ 1 - \frac{55 - 53}{55} ]}{3} \\ = 0.849665 , given\end{matrix} \\ {W}_{1} = {W}_{2} = {W}_{3} = 1. & \\ {R}_{t} = {f}_{2} ( {X}_{t} , {X}_{t - 1} ) & ( B )\end{matrix}$$

Where Xₜ and Xₜ₋₁ may refer to values of X determined at different times, for example, at the present time “t” and at a previous time “t−1”. In one example, the function f₂ may be defined in an equation below.

f₂(Xₜ,Xₜ₋₁)=Xₜ−Xₜ₋₁,−1≦f₂≦1

The value of Rₜ may therefore indicate whether an exercise performance level is improved. If Rₜ is positive, the user has made progress in performance. On the contrary, if Rₜ is negative, the user has regressed in performance.

Gₜ=f₃(Xₜ) (C)

The value of Gₜ may be related to the value of X calculated at the present time and may be used in at least one of the game-mode or share-mode operation. The function f₃ in one example may be defined in an equation below.

f₃(Xₜ)=100Xₜ

For example, Gₜ=100×0.849665=84.9665.

Cₜ=f₄(X_{i,t},X_{j,t}) (D)

Where Cₜ may refer to a comparison between the values of X achieved by two users “i” and “j” at the present time and may be used in at least one of the game-mode or share-mode operation. The function f₄ in one example may be defined in an equation below.

f₄(X_{i,t},X_{j,t})=100(X_{i,t}−X_{j,t})

For example, Cₜ=100×(0.849665−0.80334)=4.6324.

In the present example, Y̅ may be stored in the second section 23-2 as the physiological data, S̅, X, Xₜ, Xₜ₋₁, Rₜ, Gₜ and Cₜ may be stored in the second section 23-2 as the state data, and the functions f₁, f₂, f₃ and f₄ may be stored in the third section 23-3 as the algorithms.

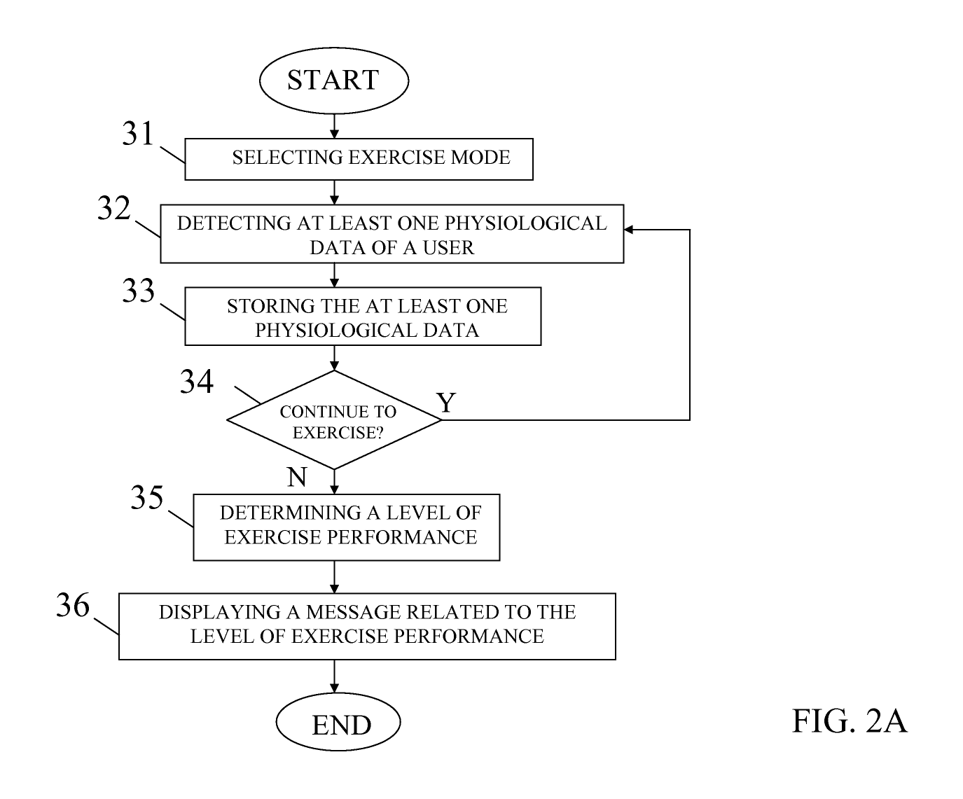

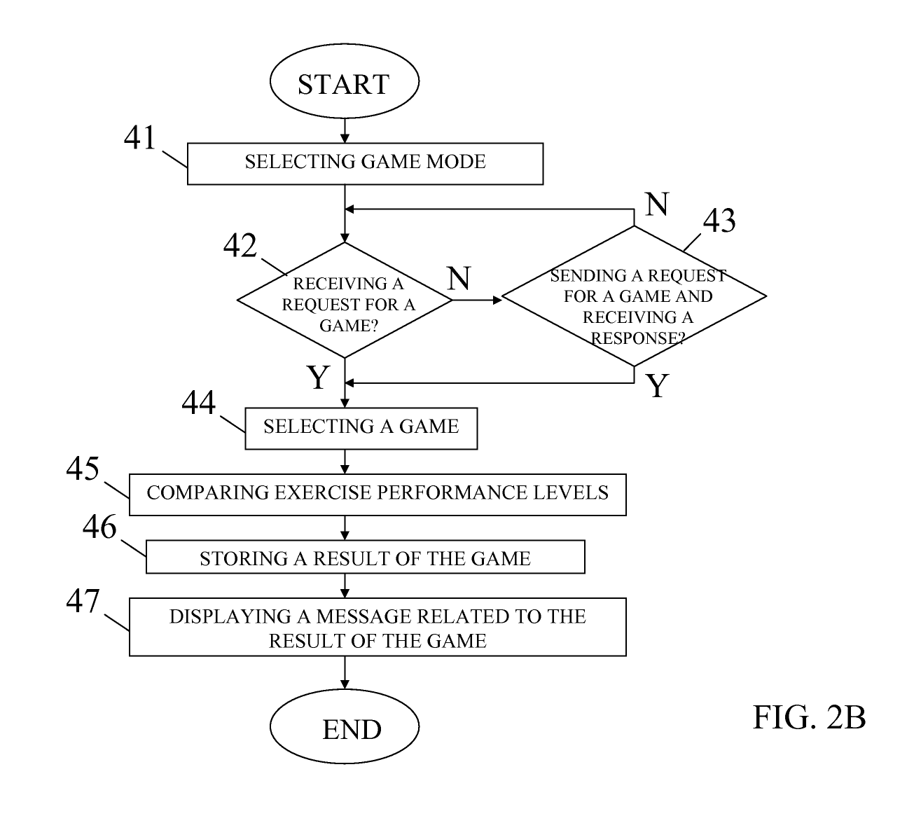

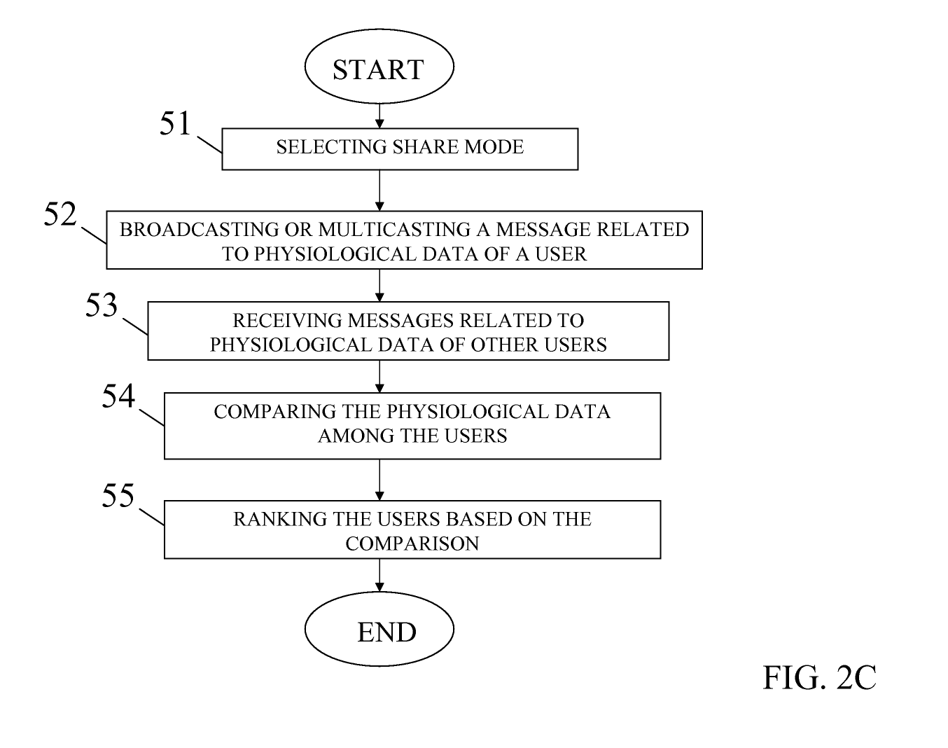

FIG. 2A is a flow diagram illustrating a first mode of the exercise assisting device 10 illustrated in FIG. 1A. Referring to FIG. 2A, the exercise assisting device 10 may operate in an exercise mode due to a user's selection at step 31. Also referring to FIGS. 1A and 1B, the detector unit 13 may detect at least one physiological data of a user while the user is exercising or immediately after his or her exercise at step 32. In one example, the detector unit 13 may be configured to measure the at least one physiological data at a predetermined period of time. The physiological data may include at least one of pulse rate, blood pressure, sweat sodium concentration, body temperature, step counts, walking, jogging speed or exercise duration. Next, at step 33, the at least one physiological data may be stored in the memory module 23 in the “physiological data” area. The detection at step 32 and the storing at step 33 may be repeated if the user continues to exercise at step 34. If the user ceases the exercise, at step 35, a level of exercise performance may be determined based on the at least one physiological data. In one example, the exercise performance level may include at least one of the above-mentioned X or Rₜ, which may be obtained by performing an algorithm in the “algorithms” area. The exercise performance level may then be stored in the memory module 23 in the “state data” area. Next, a message related to the exercise performance level, which may include an image or text or both, audio or video, may be displayed at the output unit 11 at step 36.

FIG. 2B is a flow diagram illustrating a second mode of the exercise assisting device 10 illustrated in FIG. 1A. Referring to FIG. 2B, the exercise assisting device 10 may operate in a game mode due to a user's selection at step 41. Also referring to FIGS. 1A and 1B, the user may receive a request for a game at step 42 sent over a communication network through the network communicating module 22. On the other hand, the user may send a request for a game at step 43. Once another user or other users agree to join the game, a game program may be selected from the memory module 23 at step 44. By performing the game program, a comparison on an exercise performance level between the user and another user or among a group of users may be made based on the values of Gₜ and Cₜ at step 45. A result of the game may be stored in the memory module 23 in the “game history data” area at step 46. Next, a message related to the result of the game may be displayed at the output unit 11 at step 47. In one example, if the user suffers consecutive losses, the images or texts displayed may be different from those displayed with respect to the first loss. Similarly, if the user enjoys consecutive wins, the images or texts displayed may be different from those displayed with respect to the first win.

FIG. 2C is a flow diagram illustrating a third mode of the exercise assisting device 10 illustrated in FIG. 1A. Referring to FIG. 2C, the exercise assisting device 10 may operate in a share mode due to a user's selection at step 51. At step 52, a user may broadcast a message related to his or her physiological data to other users, or multicast the message to a predetermined group of users. On the other hand, at step 53, the user may receive a message broadcast or multicast from other users. The physiological data may be compared among the users based on the values of Gₜ and Cₜ at step 54. Next, a ranking among the users based on the comparison may be provided at step 55 for the user's reference. The share mode may enable a user to exchange opinions with other users on an exercise scheme and tailor his or her exercise regimen. To facilitate communications in the share mode, in one example, the network communicating module 22 of the exercise assisting device 10 may be configured to support Short Message Service (SMS) and Multimedia Messaging Service (MMS) applications. SMS may refer to a service that permits the sending of short messages or text messages between mobile phones or other handheld devices. MMS may refer to a standard for telephony messaging systems that allow sending messages including multimedia objects (images, audio, video, rich text) as well as text messages as in SMS.

In describing representative examples of the present invention, the specification may have presented the method and/or process of the present invention as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process of the present invention should not be limited to the performance of their steps in the order written, and one skilled in the art can readily appreciate that the sequences may be varied and still remain within the spirit and scope of the present invention.

It will be appreciated by those skilled in the art that changes could be made to the examples described above without departing from the broad inventive concept thereof. It is understood, therefore, that this invention is not limited to the particular examples disclosed, but it is intended to cover modifications within the spirit and scope of the present invention as defined by the appended claims.

We claim:

1. An exercise assisting device comprising:
a detector configured to detect at least one physiological data of a user;
a control module configured to convert the at least one physiological data into at least one index in accordance with an algorithm, each of the at least one index being representative of an exercise performance level;
a memory module including:
a first section configured to store a number of predetermined data related to a number of exercise performance levels;
a second section configured to store the at least one index; and
a third section configured to store the algorithm; and
a display module configured to display at least one of the number of predetermined data in accordance with the at least one index.

2. The device of claim 1, wherein the number of predetermined data include at least one of images, texts, optical signals or audio signals.

3. The device of claim 1, wherein the at least one physiological data includes at least one of pulse rate, blood pressure, sweat sodium concentration, body temperature, duration time, step counts or speed rate of the user.

4. The device of claim 1, wherein the memory module further includes a fourth section configured to store at least one game program.

5. The device of claim 4 further comprising a network communicating module configured to provide communications over a communications network.

6. The device of claim 5, wherein one of the at least one game program includes a comparison of one of the at least one index with that of another user over the communications network.

7. The device of claim 5, wherein the memory module further includes a fifth section configured to store a result of a game after one of the at least one game programs is performed.

8. The device of claim 7, wherein the display module displays at least one of the number of predetermined data in accordance with the result of the game.

9. An exercise assisting device comprising:
a detector configured to detect at least one physiological data of a user;
a network communicating device configured to allow the user to play a game over a communications network, the game including a comparison based on one of the at least one physiological data with that of another user;
a memory module including:
a first section configured to store a number of predetermined data related to a number of exercise performance levels;
a second section configured to store the at least one physiological data; and
a third section configured to store a program of the game; and
a display module configured to display at least one of the number of predetermined data in accordance with a result of the game.

10. The device of claim 9 further comprising a control module configured to convert the at least one physiological data into at least one index in accordance with an algorithm, each of the at least one index being representative of an exercise performance level.

11. The device of claim 10, wherein the memory module further includes a fourth section configured to store the at least one index.

12. The device of claim 11, wherein each of the at least one index is representative of an exercise performance level, and the display module displays at least one of the number of predetermined data in accordance with the at least one index.

13. The device of claim 10, wherein the control module calculates the at least one index based on a set of physiological data detected by the detector.

14. The device of claim 13, wherein the set of physiological data including at least one of pulse rate, blood pressure, sweat sodium concentration, body temperature, duration time, step counts or speed rate of the user

15. The device of claim 13, wherein the set of physiological data including at least one of maximum heart rate, exercise duration time and sweat sodium concentration.

16. The device of claim 10, wherein the control module calculates the at least one index based on a set of reference data related to intensity and duration of exercise.

17. The device of claim 16, wherein the set of reference data includes maximum heart rate, exercise duration time and sweat sodium concentration.

18. The device of claim 10, wherein the control module calculates the at least one index based on a set of physiological data detected by the detector and a set of reference data related to intensity and duration of exercise.

19. An exercise assisting device comprising:
a detector configured to detect at least one physiological data of a user;
a control module configured to convert the at least one physiological data into at least one index in accordance with an algorithm, each of the at least one index being representative of an exercise performance level;
a network communicating device configured to allow the user to play a game over a communications network, the game including a comparison based on one of the at least one index with that of another user;
a memory module including:
a first section configured to store a number of predetermined data related to a number of exercise performance levels;
a second section configured to store the at least one index and a result of the game; and
a third section configured to store a program of the game; and
a display module configured to display at least one of the number of predetermined data in accordance with at least one of the at least one index or the result of the game.

20. The device of claim 19, wherein the at least one physiological data includes at least one of pulse rate, blood pressure, sweat sodium concentration, body temperature, exercise duration time, step counts or speed rate of the user.
